(19) United States
(12) Patent Application Publication
(10) Pub. No.: US 2010/0240972 A1
(43) Pub. Date: Sep. 23, 2010

(54) Slider Spot Check Pulse Oximeter

(75) Inventor: Jon Neal, Parker, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/408,171

(22) Filed: Mar. 20, 2009

(51) Int. Cl. A61B 5/1455; H01R 43/00

(52) U.S. Cl. 600/324; 29/592.1

(57) ABSTRACT

A slider spot check pulse oximeter may include a first portion and a second portion. The first portion may include a sensor configured to monitor physiological parameters of a patient. The second portion may include a display configured to display the monitored physiological parameters. The second portion may be configured to slide relative to the first portion such that the second portion substantially exposes the sensor when in an open position and substantially covers the sensor when in a closed position.

### BACKGROUND

The present disclosure relates generally to medical monitoring devices and, more particularly, to pulse oximeters.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood (SpO₂), the volume of individual blood pulsations supplying the tissue, and/or the rate of blood pulsations corresponding to each heartbeat of a patient.

Pulse oximeters typically utilize a non-invasive sensor that is placed on or against a patient's tissue that is well perfused with blood, such as a patient's finger, toe, forehead or earlobe. The pulse oximeter sensor emits light and photoelectrically senses the absorption and/or scattering of the light after passage through the perfused tissue. The data collected by the sensor may then be used to calculate one or more of the above physiological characteristics based upon the absorption or scattering of the light. More specifically, the emitted light is typically selected to be of one or more wavelengths that are absorbed and/or scattered in an amount related to the presence of oxygenated versus de-oxygenated hemoglobin in the blood. The amount of light absorbed and/or scattered may then be used to estimate the amount of oxygen in the tissue using various algorithms.

Pulse oximeters and other medical devices are typically mounted on stands that are positioned adjacent to a patient's bed or an operating room table. When a caregiver desires to command the medical device (e.g., program, configure, and so-forth), the caregiver may manipulate controls or push buttons on the monitoring device itself. The monitoring device typically provides results or responses to commands on a Liquid Crystal Display (“LCD”) screen mounted in an externally visible position on the medical device. Patient data, alerts, and other information may be displayed on the monitor directly, or may be transmitted to a central computer monitored by caregivers.

However, in certain situations it may be desirable to have a pulse oximeter that is small, lightweight, inexpensive and battery operated. For example, conventional monitors may be too heavy and bulky to be moved from one patient to another when only periodic patient monitoring is desired. Furthermore, when medical treatment is desired in a remote location, access may not be available to a conventional power source. Therefore, smaller, battery-operated pulse oximeters may be used in such situations.

Hand-held oximeters, commonly referred to as spot check pulse oximeters, are typically found in two varieties. The first variety employs a transmittance-type sensor in which an emitter and a detector are positioned on opposite sides of a patient's finger, for example. These devices generally include a first portion and a second portion biased toward each other with a spring. A display is typically housed in the first portion to provide a patient or clinician with physiological data. The device is attached to a finger by applying a counter force to the spring to separate the two portions to allow the oximeter to be clipped onto the finger. One disadvantage of this configuration is that finger attachment requires two hands. A first hand is needed to separate the two portions, while the device is attached to a finger of the second hand.

The second variety of spot check pulse oximeters addresses this issue by employing a reflectance-type pulse oximetry sensor in which the emitter and detector are located on the same side of the oximeter. In this configuration, single-handed operation is possible because the patient merely has to place a finger on the sensor. However, one disadvantage of this configuration is that the sensor is exposed when not covered by the finger, making the sensor susceptible to contamination by dirt or other debris that may adhere to the sensor and interfere with light transmission. Furthermore, pulse oximeters of this type expose the sensor to abrasion during transport. For example, if the oximeter is placed in a pocket when not in use, the reflectance-type sensor may become scratched by other items within the pocket. Scratches on the surface of the sensor may interfere with light transmission and result in inaccurate readings. Therefore, it is desirable to have a spot check pulse oximeter that may be operated with a single hand and configured to protect the sensor when not in operation.

### SUMMARY

Certain aspects commensurate in scope with certain disclosed examples are set forth below. It should be understood that these aspects are presented merely to provide the reader with a brief summary of certain embodiments and that these aspects are not intended to limit the scope of the disclosure or the claims. Indeed, the disclosure and claims may encompass a variety of aspects that may not be set forth below.

Some embodiments described herein are directed to a pulse oximeter including a first portion that includes at least one light emitter and at least one light detector and a second portion that includes a display adapted to output physiological data. The emitters and detectors may be capable of acquiring physiological data from a patient's finger, for example. The pulse oximeter also may include a drive engine, an oximetry engine, and a slider mechanism. The slider mechanism may be disposed between the first portion and the second portion and configured to facilitate translation of the second portion relative to the first portion.

Other embodiments described herein are directed to a hand-held pulse oximeter that may include a first portion having a reflectance-type pulse oximetry sensor being capable of communicatively coupling to a patient's finger, for example. The hand-held pulse oximeter also may include a pulse oximetry circuit and a second portion that may include a display adapted to receive and display physiological data. The second portion may be capable of translation relative to the first portion between a closed position and an open position.

Further embodiments described herein are directed to a method of manufacturing a hand-held pulse oximeter that may include providing a pulse oximetry sensor and disposing the pulse oximetry sensor on a first portion of the hand-held pulse oximeter. The method also may include providing a display and disposing the display on a second portion of the hand-held pulse oximeter. The method may further include securing the first portion to the second portion such that the second portion is capable of translation relative to the first portion.

### BRIEF DESCRIPTION OF THE DRAWINGS

Advantages of the disclosed embodiments may become apparent upon reading the following detailed description and upon reference to the drawings in which:

FIG. 1 is a perspective view of a slider pulse oximeter in an open position in accordance with an embodiment;

FIG. 2 is a perspective view of the slider pulse oximeter of FIG. 1 in a closed position in accordance with an embodiment;

FIG. 3 is a side view of the slider pulse oximeter of FIG. 1 with a belt clip in accordance with an embodiment;

FIG. 4 is a block diagram of the slider pulse oximeter of FIG. 1 in accordance with an embodiment;

FIG. 5 is a cutaway top view of the slider pulse oximeter of FIG. 1 in an open position in accordance with an embodiment;

FIG. 6 is a cross-sectional side view of the slider pulse oximeter of FIG. 1 taken along the 6-6 line of FIG. 5 in accordance with an embodiment;

FIG. 7 is a cross-sectional front view of the slider pulse oximeter of FIG. 1 taken along the 7-7 line of FIG. 5 in accordance with an embodiment;

FIG. 8 is a perspective view of a slider mechanism that may be used in the slider pulse oximeter of FIG. 1 in accordance with an embodiment;

FIG. 9 is a cutaway top view of the slider pulse oximeter of FIG. 1 between an open and closed position in accordance with an embodiment; and

FIG. 10 is a cross-sectional front view of the slider pulse oximeter of FIG. 1 taken along the 10-10 line of FIG. 9 in accordance with an embodiment.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The presently disclosed embodiments are directed toward a self-contained, hand-held pulse oximetry system that is small and lighweight such that it can be carried by a patient or clinician. The pulse oximetry system may include a reflectance-type pulse oximetry sensor within a first portion and a display within a second portion. The second portion may be configured to slide relative to the first portion between an open and a closed position. In the open position, the sensor may be exposed such that a patient's finger may be placed on the sensor. The pulse oximetry system may then compute blood-oxygen saturation and/or heart rate and display these parameters on the display. After use, the patient or clinician may slide the second portion into a closed position such that the second portion substantially covers the sensor. In this manner, the pulse oximetry system may be transported while protecting the sensor from scratches, dirt and/or other contaminants. For example, the sensor may remain substantially clean and unmarred even when carried in a pocket of the patient or clinician.

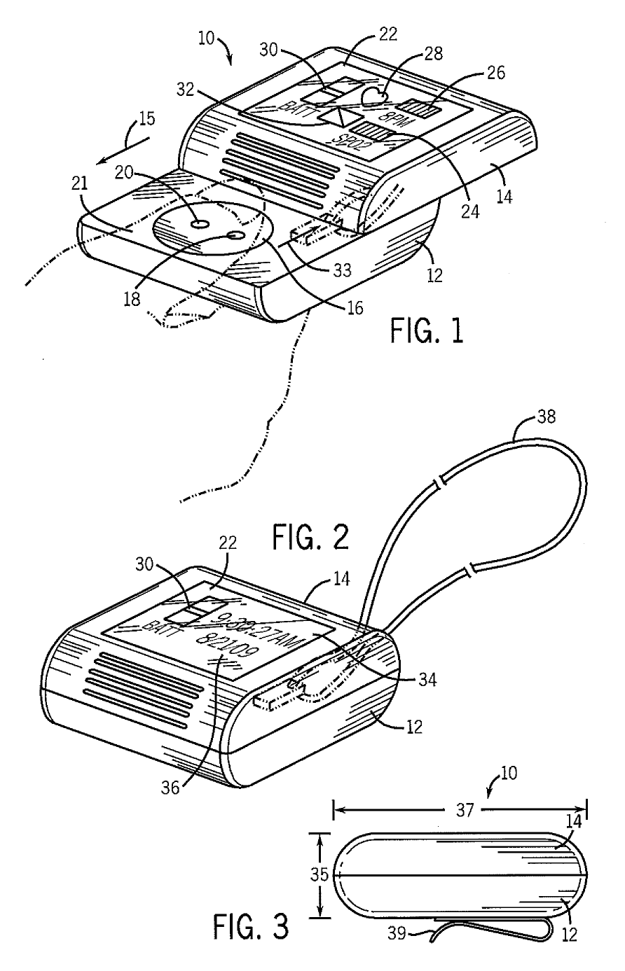

To prevent sensor contamination, in accordance with one embodiment, a cover may be placed over the sensor when the pulse oximeter is not in use. FIG. 1 is a perspective view of a slider pulse oximeter 10 consistent with this configuration. As illustrated, the slider pulse oximeter 10 includes a first portion 12 and a second portion 14. The second portion 14 is configured to slide in a direction 15 to cover a sensor membrane 16 such that the sensor membrane 16 may remain substantially free of contamination when the slider pulse oximeter 10 is not in use. The first portion 12 also includes an emitter 18 and a detector 20 located underneath the sensor membrane 16. In this embodiment, the emitter 18 and detector 20 are components of a reflectance-type pulse oximetry sensor. The sensor membrane 16 may be composed of a transparent material (e.g., glass or plastic) configured to facilitate light passage between the emitter 18, the detector 20, and a patient's finger 21. As discussed in detail below, a patient places a finger 21 on the sensor membrane 16. Light from the emitter 18 is then reflected/scattered by the finger 21 and detected by the detector 20. In this manner, various physiological parameters of the patient may be measured.

Reflectance-type sensors operate by emitting light into the tissue and detecting the light that is transmitted and scattered by the tissue. Reflectance-type sensors include an emitter 18 and detector 20 that are typically placed on the same side of a sensor site. For example, a reflectance-type sensor may be placed on a patient's fingertip 21 such that the emitter 18 and detector 20 lie side-by-side. Reflectance-type sensors detect light photons that are scattered back to the detector 20. During operation, the emitter 18 directs one or more wavelengths of light onto the patient's fingertip 21, and the light received by the detector 20 is processed to determine various physiological characteristics of the patient. In each of the embodiments discussed herein, it should be understood that the locations of the emitter 18 and detector 20 may be interchanged. Regardless of the arrangement, the slider pulse oximeter 10 will perform in substantially the same manner.

The emitter 18 and the detector 20 may be of any suitable type. For example, the emitter 18 may be one or more light emitting diodes adapted to transmit one or more wavelengths of light in the red to infrared range, and the detector 20 may be one or more photodetectors selected to receive light in the range or ranges emitted from the emitter 18. Alternatively, the emitter 18 may also be a laser diode or a vertical cavity surface-emitting laser (VCSEL). Emitter 18 and detector 20 may also include optical fiber elements. An emitter 18 may include a broadband or “white light” source, in which case the detector 20 could include any variety of elements for selecting specific wavelengths, such as reflective or refractive elements or interferometers. These kinds of emitters and detectors would typically be coupled to the rigid or rigidified sensor via fiber optics. Alternatively, the slider pulse oximeter 10 may sense light detected from the tissue at a different wavelength from the light emitted into the tissue. Such sensors may be adapted to sense fluorescence, phosphorescence, Raman scattering, Rayleigh scattering and multi-photon events or photoacoustic events. Similarly, in other applications, a tissue water fraction (or other tissue constituent related metric) or a concentration of one or more biochemical components in an aqueous environment may be measured using two or more wavelengths of light. In certain embodiments, these wavelengths may be infrared wavelengths between about 1,000 nm to about 2,500 nm.

It should be understood that, as used herein, the term “light” may refer to one or more of infrared, visible or ultraviolet and may also include any wavelength within the infrared, visible or ultraviolet spectra, and that any suitable wavelength of light may be appropriate for use with the present techniques.

Returning to FIG. 1, the illustrated embodiment includes a display 22 disposed within the second portion 14 of the slider pulse oximeter 10. The display 22 in this embodiment may be configured to display graphical and/or textual information to a patient or clinician. For example, the display 22 may be an LCD display, an organic light emitting diode (OLED) display, or the like. In the illustrated embodiment, the display 22 includes a numerical representation of blood-oxygen saturation (SpO₂) 24. The SPO₂ value may be expressed in terms of a oxygen saturation percentage, for example. The display 22 also may include a numerical representation of heart rate 26 expressed in terms of beats per minute (BPM). Furthermore, the display 22 may include a graphical heart 28 that illuminates upon detection of a patient's heart beat. This symbol 28 presents the patient or clinician with a graphical representation of the patient's pulse. In addition, the display 22 may include a graphical representation of remaining battery life 30. For example, as illustrated, the battery life indicator 30 includes three bars, each representing a third of the remaining battery life. In other embodiments, more or fewer bars may be employed to indicate the remaining battery life.

The display 22 also may include an information window 32 that may present the patient or clinician with information about the condition of the patient or slider pulse oximeter 10. For example, the window 32 may display a graphical icon indicating an excessively low blood-oxygen saturation. In such an embodiment, the slider pulse oximeter 10 may be programmed with a threshold blood-oxygen saturation. If a patient's blood-oxygen saturation drops below this threshold value, the window 32 may display an icon indicative of that condition to warn the patient or clinician. Similarly, the window 32 may display an icon indicative of excessively high or low pulse rate, a steady decline in blood-oxygen saturation, a sudden drop in blood-oxygen saturation, or other detected conditions. Furthermore, the window 32 may display an icon indicative of a condition of the slider pulse oximeter 10. For example, the window 32 may display an icon indicating a sensor failure and/or improper contact between the finger 21 and the sensor, among other conditions. In addition, the window 32 may display a textual message representative of any of the above conditions, alone or in combination with an icon.

Other display configurations may be employed in alternative embodiments. For example, the display 22 may include additional graphical or textual information (e.g., a graph of heart rate as a function of time). Conversely, the display may include fewer elements, such as a numerical representation of heat rate and blood-oxygen saturation alone. Other embodiments may include a series of LEDs instead of a graphical display. For example, the display 22 may include an LED indicative of a low blood-oxygen saturation and another LED indicative of an excessive heart rate. Further embodiments may include an LED that illuminates upon detection of a heart beat, similar to the previously described heart icon 28. Certain embodiments may include multicolored LEDs to indicate various physiological conditions. For example, a green LED may illuminate upon detection of a heart beat, while a red LED may illuminate upon detection of a low blood-oxygen saturation.

Additionally, although not depicted, embodiments may include one or more pushbuttons coupled to the first portion 12 and/or second portion 14. The pushbuttons may allow a patient or clinician to activate and/or deactivate the slider pulse oximeter 10 and/or change the configuration of the display 22. For example, a pushbutton may enable a patient or clinician to cycle through various physiological data displayed on the display 22. Furthermore, the pushbuttons may enable a patient or clinician to input range limits for various physiological parameters. For example, the patient or clinician may enter a maximum heart rate and/or a minimum blood-oxygen saturation. If the slider pulse oximeter 10 detects a physiological parameter outside of the input range, the display 22 may inform the patient or clinician of the detected condition. Furthermore, as described below, the slider pulse oximeter 10 may emit an audible alarm if a physiological parameter exceeds the input range.

The slider pulse oximeter 10 of the present embodiment is configured to facilitate single-handed operation. For example, as seen in FIG. 1, the slider pulse oximeter 10 may be placed between a patient's thumb 21 and index finger. The patient may then slide the second portion 14 relative to the first portion 12 in a direction 33 with the thumb 21, thereby exposing the sensor membrane 16 of the first portion 12. The patient may then place the thumb 21 onto the sensor membrane 16 to obtain a measurement of the patient's physiological data. The physiological data may be displayed as a graphical and/or numeric representation on the display 22. Once a measurement has been taken, the patient may close the slider pulse oximeter 10 by sliding the second portion 14 over the sensor membrane 16 in direction 15 with the thumb 21 or index finger. In this manner, a patient may operate the slider pulse oximeter 10 with a single hand.

One-handed operation is particularly helpful when a patient desires to measure blood-oxygen saturation and/or pulse while engaged in another activity. For example, if a patient desires to measure physiological parameters while running or otherwise exercising, the patient may draw the slider pulse oximeter 10 from a pocket, for example. The patient may then open the slider pulse oximeter 10 single-handedly, take a measurement, and then close the slider pulse oximeter 10. In this manner, the patient may take the desired measurements without significantly interfering with the activity.

Similarly, a clinician may operate the slider pulse oximeter 10 with a single hand. The clinician may open the slider pulse oximeter 10 in a similar manner to the patient. Then, the clinician may place a patient's finger on the sensor membrane 16 to measure a patient's physiological parameters. Single-handed operation of the slider pulse oximeter 10 may reduce patient monitoring time, thereby increasing clinician efficiency.

To protect the sensor when the slider pulse oximeter 10 is not in use, the second portion 14 may slide in the direction 15 to cover the sensor membrane 16, as shown in FIG. 2. FIG. 2 presents the slider pulse oximeter 10 in a closed position in which the sensor membrane 16 is substantially covered by the second portion 14. This configuration may prevent dirt and other contaminants from adhering to the surface of the sensor membrane 16 and interfering with operation of the slider pulse oximeter 10.

The display 22, shown in FIG. 2, presents a different interface than the interface described above with regard to FIG. 1. As appreciated, while the slider pulse oximeter 10 is in the closed position, a finger may not be placed on the sensor membrane 16. This limitation effectively disables operation of the slider pulse oximeter 10. Therefore, displaying patient physiological data on the display 22 may be superfluous. However, instead of displaying a blank screen, the display 22 may provide the patient or clinician with other useful information. For example, the display 22 of the illustrated embodiment includes a battery indicator 30, a time 34, and a date 36. Other information may also be provided to the patient or clinician in alternative embodiments (e.g., timer, patient information, etc.). In addition, the slider pulse oximeter 10 may emit an audible reminder at a desired time. For example, a clinician may input a time to begin rounds, i.e., examining patients with the slider pulse oximeter 10. At the appropriate time, the slider pulse oximeter 10 may emit an audible reminder to inform the clinician to begin rounds.

To conserve power, the slider pulse oximeter 10 may deactivate the electronic components associated with measurement of patient physiological data while the slider pulse oximeter 10 is in the closed position. For example, the slider pulse oximeter 10 may disable the emitter 18 and detector 20. In alternative embodiments, the slider pulse oximeter 10 may also deactivate the display 22 while the slider pulse oximeter 10 is in the closed position to further reduce power consumption. In further embodiments, other techniques for activating and deactivating the slider pulse oximeter 10 may be included. For example, the slider pulse oximeter 10 may include a power button or switch, or, alternatively, the slider pulse oximeter 10 may include a pressure sensitive button embedded within the sensor membrane 16 that activates the slider pulse oximeter 10 upon contact with a patient's finger 21. In addition, the slider pulse oximeter 10 may be configured to deactivate the electronic components upon detecting an absence of the finger 21 for a predetermined time. For example, if the slider pulse oximeter 10 is in the open position, but no finger 21 has contacted the emitter 18 and detector 20 for two minutes, for example, the slider pulse oximeter 10 may be deactivated.

To facilitate transport, the slider pulse oximeter 10 may include a lanyard 38 disposed to the first portion 12. For example, the lanyard 38 may be worn around the clinician's neck to provide easy access to the slider pulse oximeter 10 as the clinician examines each patient. Similarly, the lanyard 38 may be worn around the patient's neck to provide easy access to the slider pulse oximeter 10 whenever the patient desires to monitor blood-oxygen saturation and/or heart rate. As shown in FIG. 3, alternative embodiments of the slider pulse oximeter 10 may include a belt clip 39 disposed to the first portion 12 and configured to secure to the belt of a patient or clinician. In each of these embodiments, the second portion 14, while in the closed position, may protect the sensor membrane 16 from becoming scratched or otherwise contaminated during transport. As FIG. 3 illustrates, the slider pulse oximeter 10 may have a low profile in the closed position to further facilitate transportation. For example, a thickness 35 of the slider pulse oximeter 10 may be approximately 1-3 cm in certain embodiments. In addition, the slider pulse oximeter 10 may have a length 37 of about 4-7 cm. This small size may enhance the portability of the slider pulse oximeter 10.

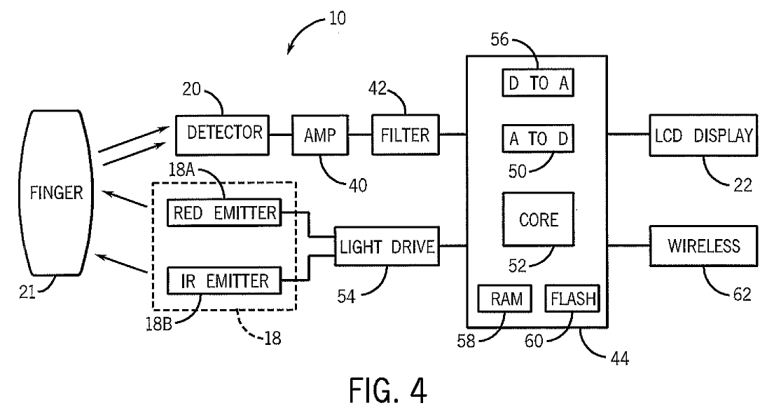

Turning now to FIG. 4, a block diagram of a slider pulse oximeter 10 is illustrated in accordance with an embodiment. It will be understood that an actual implementation may include more or fewer components as needed for a specific application. In this embodiment the slider pulse oximeter 10 may include a red emitter 18A and an infra-red emitter 18B that are configured to transmit electromagnetic radiation through the tissue of the patient's finger 21. In accordance with this embodiment, the emitters 18A and 18B may include respective LEDs that emit electromagnetic radiation in the respective region of the electromagnetic spectrum. The emitted radiation transmitted from the emitters 18A and 18B into a patient's tissue is detected by the detector 20 after the radiation has passed through blood perfused tissue of the finger 21. The detector 20 generates a photoelectrical signal correlative to the amount of radiation detected.

The signal generated by the detector 20 may then be amplified by an amplifier 40, filtered by a filter 42, and provided to one or more processor(s) 44. The processor(s) 44 may include an analog-to-digital converter 50 that converts the analog signal provided by the detector 20 into a digital signal. The analog-to-digital converter 50 may provide the digital signal to a core 52 to be processed for computing physiological parameters related to the patient. For example, the core 52 may compute a percent oxygen saturation of hemoglobin and/or a pulse rate, among other useful physiological parameters, as will be appreciated by one of ordinary skill in the art. By utilizing an analog-to-digital converter 50 within the processor(s) 44, the size and cost of the oximeter may be reduced, compared to traditional pulse oximeters that use a separate analog-to-digital converter. In presently contemplated embodiments, the processor(s) 44 may include a Mixed-Signal Microcontroller such as model number C8051F353 available from Silicon Laboratories.

In addition to computing physiological parameters, the processor(s) 44 may control the timing and intensity of the emitted electromagnetic radiation of the emitters 18A and 18B via a light drive circuit 54. In embodiments, the light drive circuit 54 may be driven by a digital-to-analog converter 56, included in the processor(s) 44. By utilizing a digital to analog converter 56 within the microprocessor 44, the size and cost of the oximeter may be reduced, compared to traditional pulse oximeters that use a separate digital-to-analog converter. In accordance with an embodiment, the light drive circuit 54 may have a low part count such as the light drive circuit discussed in detail in U.S. patent application Ser. No. 12/343,799, entitled “LED Drive Circuit and Method for Using Same” which was filed Dec. 24, 2008, and is incorporated herein by reference in its entirety for all purposes. The reduced part count of the drive circuit 54 may further reduce the size, complexity, and cost of the slider pulse oximeter 10.

Furthermore, the processor(s) 44 may also include a RAM 58 and/or a flash memory 60 coupled to the core processor 52. The RAM 58 may be used to store intermediate values that are generated in the process of calculating patient parameters. The flash memory 60 may store certain software routines used in the operation of the slider pulse oximeter 10, such as measurement algorithms, LED drive algorithms, and patient parameter calculation algorithms, for example. In certain embodiments, the slider pulse oximeter 10 may include simplified pulse oximetry algorithms such that the computer code associated with those algorithms may be contained in the memory components of the processor(s) 44,

In some embodiments, the slider pulse oximeter 10 may also include other memory components that are not included in the processor(s) 44. For example, the slider pulse oximeter 10 may include a read-only memory (ROM), which may be used to store such things as operating software for the slider pulse oximeter 10 and algorithms for computing physiological parameters. In other embodiments, however, all of the processing memory and measurement software is included in the processor(s) 44.

Furthermore, in some embodiments, the slider pulse oximeter 10 may also include a long-term memory device used for long-term storage of measured data, such as measured physiological data or calculated patient parameters. In other embodiments, however, the long-term memory device may be omitted to reduce the cost and/or part count of the slider pulse oximeter 10. By omitting the long-term memory device, smaller, less expensive memory components may be utilized, thereby reducing the part count and the size and complexity of the slider pulse oximeter 10, compared to traditional pulse oximetry systems.

Further embodiments may include a memory card reader (not shown) configured to electrically couple with a removable memory card (not shown). The memory card may include patient identification information. This information may be uploaded to the processor(s) 44 automatically upon insertion of the memory card. In addition, the memory card may be configured to store measured data, such as measured physiological data or calculated patient parameters. In certain embodiments, the measured data may be associated with the patient identification information stored on the memory card. The stored data may be transferred to a computer, for example, by removing the memory card from the slider pulse oximeter 10 and inserting it into a reader electrically coupled to the computer.

As mentioned previously, also included in the slider pulse oximeter 10 is a display that may be coupled to the processor(s) 44 to allow for display of the computed physiological parameters. For example, the display may include an LCD display 22, which is operably coupled to the processor(s) 44 and programmed to operate as described above in relation to FIGS. 1 and 2. The LCD display 22 may include drive circuitry configured to convert the processor 44 output into a format suitable for driving the LCD display 22.

Embodiments may also include a wireless device 62 configured to transmit computed patient parameters such as, for example, pulse rate, blood-oxygen saturation, or the raw data. The wireless device 62 may include any suitable wireless technology. For example, the slider pulse oximeter 10 may transmit data via a wireless communication protocol such as WiFi, Bluetooth or ZigBee.

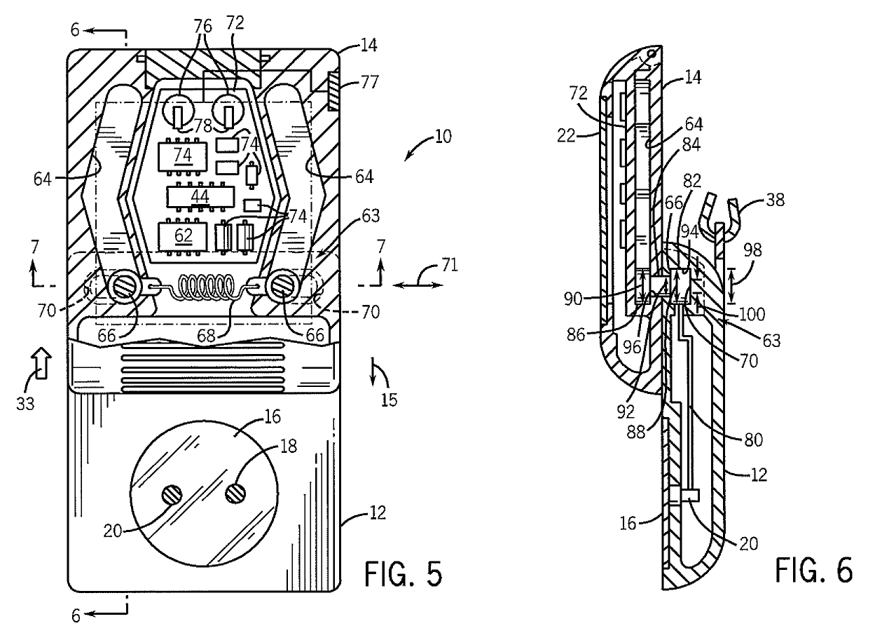

The slider pulse oximeter 10 may utilize a slider mechanism 63 to facilitate translation of the second portion 14 relative to the first portion 12. A spring-biased slider mechanism 63 is shown in the cutaway top view of FIG. 5. As appreciated, other slider mechanism configurations may be utilized in alternative embodiments. The illustrated spring-biased mechanism 63 is configured to bias the second portion 14 toward the closed position while the second portion 14 is closer to the closed position than the open position. As a patient or clinician translates the second portion 14 in direction 33, the slider mechanism 63 transitions to bias the second portion 14 toward the open position when the second portion 14 is closer to the open position than the closed position. This configuration facilitates maintaining the second portion 14 in an open position during use, and a closed position during storage and transport.

As discussed in detail below, the slider mechanism 63 of the present embodiment includes a pair of tracks 64, a pair of pins 66, a spring 68, and a pair of grooves 70. The tracks 64 are disposed within the second portion 14 and configured to facilitate translation of the second portion 14 with respect to the first portion 12. Pins 66 are disposed within the tracks 64 of the second portion 14 and the grooves 70 of the first portion 12. The pins 66 serve to secure the second portion 14 to the first portion 12, while enabling translation. Specifically, as the second portion 14 translates with respect to the first portion 12, the tracks 64 translate relative to the pins 66. Due to the chevron shape of the tracks 64, the pins 66 are driven to translate along a lateral axis 71 as the tracks 64 move along the pins 66. The grooves 70 are elongated to enable the pins 66 to translate along the lateral axis 71. Spring 68 serves to bias the pins 66 laterally inward such that the second portion 14 is biased toward the closed position when the second portion 14 is closer to the closed position. Similarly, the spring 68 serves to bias the second portion 14 toward the open position when the second portion 14 is closer to the open position.

As illustrated, the second portion 14 includes a circuit board 72. The circuit board 72 may be particularly shaped to fit between the tracks 64 to prevent interference with the slider mechanism 63. As appreciated, the circuit board 72 may be enclosed within the first portion 12 in alternative embodiments. The circuit board 72 may include the processor 44, as well as other circuit components 74, such as the circuit components discussed above with regard to FIG. 4. The circuit board 72 may also include one or more batteries 76. The batteries 76 may be any small, lightweight battery such as a “coin cell” or “button cell.” In some embodiments, the batteries 76 may be lithium ion batteries. In yet other embodiments, the batteries 76 may be nanowire batteries, i.e., high performance lithium ion batteries made from silicon nanowires. The batteries 76 serve to power the circuitry of the slider pulse oximeter 10 and are coupled to the circuitry through contacts 78. Alternative embodiments may include batteries located in the first portion 12 of the slider pulse oximeter 10. These batteries (e.g., AAA, AA, rechargeable, etc.) may be electrically coupled to the circuit board 72 by a cable extending from the first portion 12 to the second portion 14.

The second portion 14 may also include a battery door (not shown), facilitating access to the batteries 76. For example, a section of the second portion 14 may swing or slide open to expose a battery compartment, allowing batteries 76 to be changed. Alternatively, the battery door may be disposed to the first portion 12 in embodiments in which the first portion 12 houses the batteries 76. Further embodiments having non-removable batteries 76 may omit the battery door. For example, embodiments employing rechargeable batteries 76 may include a power connector coupled to the second portion 14 or the first portion 12 to facilitate recharging the batteries 76.

The illustrated embodiment also includes a wireless device 62 on the circuit board 72. As discussed above, the wireless device 62 may allow the slider pulse oximeter 10 to transmit data wirelessly to a remote monitor. As such, the wireless device 62 may include wireless transmitter circuitry and a radio frequency antenna, such as, for example a microstrip or patch antenna.

Furthermore, embodiments may also include a speaker 77 and supporting circuitry configured to drive the speaker 77. The speaker 77 may emit sound to communicate physiological data to a patient or clinician. For example, the speaker 77 may be configured to emit a beeping sound corresponding to the heartbeat of a patient, or the speaker 77 may be configured to sound an audible alarm when the patient's blood-oxygen saturation level and/or pulse falls outside of a certain acceptable range. Furthermore, as previously discussed, the speaker 77 may emit an audible reminder to a clinician to being rounds.

If the emitter 18 and detector 20 and the circuit board 72 are on different portions of the slider pulse oximeter 10, the slider pulse oximeter 10 may be configured to maintain a connection between the emitter 18 and detector 20 and the circuit board 72 throughout the range of motion of the second portion 14. As illustrated in FIG. 6, a cross-sectional side view of the slider pulse oximeter 10 taken along the 6-6 line of FIG. 5, a cable 80 may be used to electrically couple the emitter 18 and detector 20 to the circuit board 72. Specifically, one end of the cable 80 is coupled to the emitter 18 and detector 20 within the first portion 12 and the other end of the cable 80 is connected to the circuit board 72 within the second portion 14. The cable 80 may be routed through pin 66 via an internal passage. The length of cable 80 may be configured to accommodate the varying distance between the pin 66 and the circuit board 72. In this configuration, the connection between the emitter 18 and detector 20 and the circuit board 72 may be maintained as the second portion 14 translates relative to the first portion 12.

As previously discussed, the pins 66 are configured to secure the first portion 12 to the second portion 14. As shown in FIG. 6, the pin 66 includes a first head 82, a shaft 84, and a second head 86. A diameter 88 of the first head 82 and a diameter 90 of the second head 86 may be greater than a diameter 92 of the shaft 84. In this configuration, the first head 82 is confined to groove 70 and the second head 86 is confined to tracks 64. Specifically, groove 70 includes a first portion 94 and a second portion 96. A width 98 of the first portion 94 is substantially similar to the diameter 88 of first head 82. Similarly, a width 100 of the second portion 96 is substantially similar to the diameter 92 of the shaft 84. In this configuration, the first head 82 of pin 66 is confined to the first portion 94 of groove 70. Therefore, translation of pin 66 relative to the first portion 12 is restricted to the lateral axis 71.

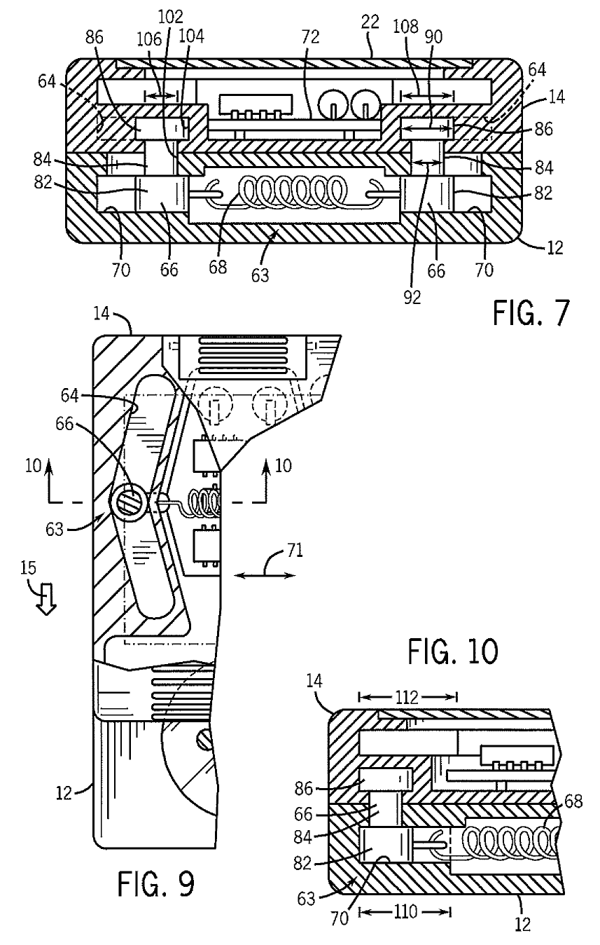

Similarly, as shown in FIG. 7, a cross-sectional front view of the slider pulse oximeter 10 taken along the 7-7 line of FIG. 5, tracks 64 may include a first portion 102 and a second portion 104. A width 106 of the first portion 102 is substantially similar to the diameter 92 of shaft 84. Furthermore, a width 108 of the second portion 104 is substantially similar to the diameter 90 of second head 86 of pin 66. In this configuration, the second head 86 of pin 66 is confined to the second portion 104 of tracks 64. Therefore, translation of pin 66 relative to the second portion 14 is restricted to the path of tracks 64.

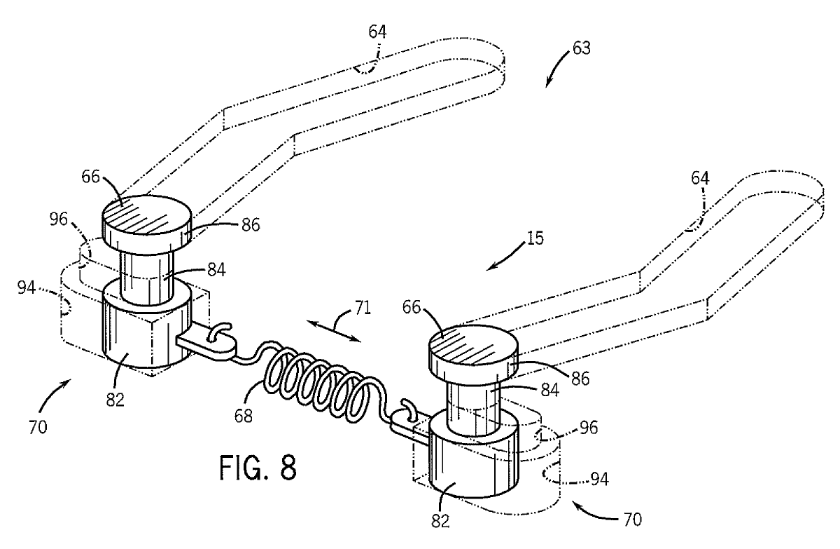

As illustrated in FIGS. 5-7, second portion 14 is biased into the open position by spring 68. Specifically, as spring 68 biases the pins 66 inward along lateral axis 71, interaction between the pins 66 and the tracks 64 urge second portion 14 toward the open position. As best seen in FIG. 8, a perspective view of the slider mechanism 63, if a patient or clinician applies a force to the second portion 14 in direction 15, tracks 64 translate in direction 15 relative to pins 66. Due to the initial laterally outward orientation of tracks 64, pins 66 are forced outward along lateral axis 71 as tracks 64 translate in direction 15. However, the spring 68 applies a counter force to pins 66 as pins 66 translate laterally outward. This counter force is experienced by the patient or clinician in the form of resistance to translation of the second portion 14 in direction 15. If the patient or clinician releases the second portion 14 while the pins 66 are within this laterally outward section of tracks 64, the second portion 14 may automatically translate back to the open position. Therefore, this configuration holds the second portion 14 in the open position during use of the slider pulse oximeter 10.

Conversely, the slider mechanism 63 is also configured to hold the second portion 14 in the closed position during transportation and storage. As illustrated in FIG. 8, tracks 64 transition from a laterally outward orientation to a laterally inward orientation at the approximate midpoint of tracks 64. Therefore, as a patient or clinician translates the second portion 14 past this transition point, the second portion 14 becomes biased toward the closed position. FIG. 9 presents a cutaway top view of the slider pulse oximeter 10 with pins 66 located at the transition point between the laterally outward and laterally inward sections of tracks 64. As illustrated, spring 68 biases pins 66 laterally inward such that the second portion 14 is biased toward an open position when the pins 66 are in the laterally outward section of tracks 64 and biased toward the closed position when the pins 66 are in the laterally inward section of tracks 64. Therefore, when pins 66 are at the transition between the two sections, the second portion 14 is not biased toward either position. However, due to the curvature of the pins 66 and the angle of the transition, the second portion 14 is unstable in this position. Consequently, any slight force applied to the second portion 14 may induce the second portion 14 to translate into the closed position or the open position.

The grooves 70 are configured to accommodate lateral translation of pins 66 as pins 66 are driven to move in the lateral direction 71 by tracks 64. As best seen in FIG. 10, a cross-sectional front view of the slider pulse oximeter 10 taken along the 10-10 line of FIG. 9, a length 110 of grooves 70 is sufficient to facilitate movement of pins 66 to an outer lateral extent substantially equal to the outer lateral extent of tracks 64. In other words, the length 110 of grooves 70 is substantially similar to a length 112 defining the inner and outer lateral extent of tracks 64. In this configuration, pins 66 may translate in lateral direction 71 as tracks 64 move in direction 15 and/or direction 33.

As a patient or clinician continues to move the second portion 14 in direction 15 past the transition point tracks 64 continue to translate in direction 15 relative to pins 66. As previously discussed, the spring 68 biases the pins 66 laterally inward. Therefore, due to the laterally inward orientation of tracks 64 past the transition point, interaction between the pins 66 and the tracks 64 induces the second portion 14 to automatically translate toward the closed position. Specifically, pins 66 apply a inward force to tracks 64 along lateral axis 71. Due to the laterally inward orientation of the tracks 64, this lateral force is converted to a force in direction 15. Consequently, if the patient or clinician releases the second portion 14 while the pins 66 are within the laterally inward section of tracks 64, the second portion 14 may automatically translate toward the closed position. Furthermore, this configuration holds the second portion 14 in the closed position during transportation and storage of the slider pulse oximeter 10.

Other slider mechanisms may be employed in alternative embodiments. For example, the slider mechanism may be configured to bias the second portion 14 toward the closed position when the second portion 14 is substantially in the closed position, and bias the second portion 14 toward the open position when the second portion 14 is substantially in the open position. In this configuration, to transition the slider pulse oximeter 10 from the closed position to the open position, the patient or clinician applies a force to the second portion 14 to overcome the bias toward the closed position After the second portion 14 translates away from the substantially closed position, the patient or clinician may translate the second portion 14 without bias toward the open position. Upon reaching the substantially open position, the second portion 14 is biased toward the open position. In this arrangement, the slider mechanism holds the second portion 14 in the open position during use, and the closed position during storage and transportation.

In a further embodiment, the slider mechanism is configured to bias the second portion 14 toward the open position, secure the second portion 14 in the closed position when the second portion 14 is substantially in the closed position, and release the second portion 14 from the closed position upon activation of a release mechanism. In other words, during transportation or storage, the second portion 14 is locked into the closed position by the release mechanism. Prior to use, the patient or clinician activates the release mechanism, thereby causing a spring to direct the second portion 14 from a closed position to an open position. The spring also serves to hold the second portion 14 in the open position during use. After use, the patient or clinician may direct the second portion 14 toward the closed position by applying a force to the second portion 14 to counteract the spring bias. Upon reaching the closed position, the release mechanism may automatically lock the second portion 14 into the closed position for transportation or storage.

Other embodiments may include a pedometer that is used to collect general information about user mobility for patients on a rehab program. Other embodiments may include memory on which music or other data may be stored. Rhythmic music may be played bio-feedback to adjust the user's pulse, etc.

Other embodiments may include reflectance sensor that includes a pressure-sensor in the pad where the finger contacts the device. The sensor may be used for open- or closed-loop feedback for ensuring the finger has the required amount of contact pressure. For example, it could sense if the patient was pressing too hard and restricting blood flow.

What is claimed is:

1. A pulse oximeter; comprising:
a first portion comprising a light emitter and a light detector, the emitter and detector capable of acquiring physiological data from a patient's finger;
a second portion comprising a display adapted to output physiological data;
a drive engine configured to activate the light emitter;
an oximetry engine configured to receive a signal corresponding to the physiological data acquired by the detector, generate an output based at least in part upon the physiological data, and transfer the output to the display; and
a slider mechanism disposed between the first portion and the second portion and configured to facilitate translation of the second portion relative to the first portion.

2. The pulse oximeter of claim 1, wherein the slider mechanism is configured to translate the second portion relative to the first portion between a closed position, in which the second portion substantially covers the at least one light emitter and the at least one light detector, and an open position, in which the second portion substantially exposes the at least one light emitter and the at least one light detector.

3. The pulse oximeter of claim 2, wherein the slider mechanism is configured to bias the second portion toward the closed position until translation of the second portion relative to the first portion exceeds a predetermined distance, and bias the second portion toward the open position in response to the second portion being translated the predetermined distance from the first portion.

4. The pulse oximeter of claim 2, wherein the slider mechanism is configured to bias the second portion toward the closed position when the second portion is substantially in the closed position, and bias the second portion toward the open position when the second portion is substantially in the open position.

5. The pulse oximeter of claim 2, wherein the slider mechanism is configured to bias the second portion toward the open position, secure the second portion in the closed position when the second portion is substantially in the closed position, and release the second portion from the closed position upon activation of a release mechanism.

6. The pulse oximeter of claim 2, wherein the drive engine and the oximetry engine are activated in response to translation of the second portion from the closed position to the open position, and deactivated in response to translation of the second portion from the open position to the closed position.

7. The pulse oximeter of claim 1, wherein the display is configured to display a numerical value indicative of blood-oxygen saturation, heart rate, respiration rate, other parameters, and/or combinations thereof.

8. The pulse oximeter of claim 1, comprising a speaker configured to emit a sound based at least in part upon the output corresponding to the physiological data, an audible reminder at a desired time, an audible alarm indicating a value of the output corresponding to the physiological data exceeds a stored value, and/or a combination thereof.

9. A hand-held pulse oximeter, comprising:
a first portion comprising a reflectance-type pulse oximetry sensor being capable of communicatively coupling to a patient's finger;
a pulse oximetry circuit adapted to receive a signal from the sensor and output physiological data of the patient; and
a second portion comprising a display adapted to receive and display the physiological data, the second portion being capable of translation relative to the first portion between a closed position and an open position.

10. The hand-held pulse oximeter of claim 9, wherein the second portion substantially covers the sensor while the second portion is in the closed position.

11. The hand-held pulse oximeter of claim 9, wherein the pulse oximetry circuit is activated in response to translation of the second portion from the closed position to the open position, and deactivated in response to translation of the second portion from the open position to the closed position.

12. The hand-held pulse oximeter of claim 9, wherein the display is configured to display a first interface while the second portion is in the closed position and a second interface, different from the first interface, while the second portion is in the open position.

13. The hand-held pulse oximeter of claim 12, wherein the first interface comprises a numerical value indicative of time, date, or a combination thereof.

14. The hand-held pulse oximeter of claim 12, wherein the second interface comprises a numerical value indicative of blood-oxygen saturation, heart rate, respiration rate, other parameters, and/or combinations thereof.

15. The hand-held pulse oximeter of claim 12, wherein the second interface comprises an indicator that activates upon detection of the patient's heart beat.

16. The hand-held pulse oximeter of claim 12, wherein the second interface comprises a text message, icon, or combination thereof, indicative of a detected condition of the patient or the hand-held pulse oximeter.

17. The hand-held pulse oximeter of claim 12, wherein the second interface comprises a graphical indication of electrical power remaining within a battery.

18. The hand-held pulse oximeter of claim 9, comprising an attachment portion capable of receiving a belt clip, a lanyard, or a combination thereof, disposed to the first portion.

19. The hand-held pulse oximeter of claim 9, wherein the pulse oximetry circuit is deactivated in response to receiving a signal indicative of an absence of a finger for a predetermined time.

20. A method of manufacturing a hand-held pulse oximeter, comprising:
providing a pulse oximetry sensor capable of sensing physiological data of a patient;
disposing the pulse oximetry sensor on a first portion of the hand-held pulse oximeter;
providing a display capable of displaying physiological data of a patient;
disposing the display on a second portion of the hand-held pulse oximeter; and
securing the first portion to the second portion such that the second portion is capable of translation relative to the first portion.
